(19) United States
(12) Patent Application Publication
(10) Pub. No.: US 2007/0185499 A1
(43) Pub. Date: Aug. 9, 2007

(54) Device for the insertion of deformable intra-ocular lenses

(75) Inventors: Rene Kammerlander, Dietikon (CH); Klaus Deinzer, Schlieren (CH)

Correspondence Address: SQUIRE, SANDERS & DEMPSEY L.L.P., 1 MARITIME PLAZA, SUITE 300, SAN FRANCISCO, CA, 94111, US

(73) Assignee: ADVANCED VISION SCIENCE, INC., Goleta, CA (US)

(21) Appl. No.: 10/590,972

(22) PCT Filed: Feb. 27, 2004
(86) PCT No.: PCT/EP04/50224

§ 371 Date: Aug. 25, 2006

(51) Int. Cl. A61F 9/00; A61F 2/16

(52) U.S. Cl. 606/107; 623/6.12

(57) ABSTRACT

The invention relates to a device that permits an intra-ocular lens, which has been elastically deformed, to be injected through a cannula (5) into an eye by means of a ram (6) with a translatory displacement action. The device contains a lens holder (1), which can be inserted into said device. The lens holder contains an elastic base (8), which is deformable in such a way that it can be converted from a relaxed, open position into a tensioned, closed position by bending. During said deformation, the intra-ocular lens that is in contact with the lens holder (1) is subjected to an increased curvature. This permits the careful deformation of the intra-ocular lens and a precise, reliable injection of the latter.

### FILED OF THE INVENTION

The invention relates to a device for the insertion of deformable intra-ocular lenses, with which device an intra-ocular lens in an elastically deformable state can be injected through a cannula into an eye by means of a plunger which can be moved in a translatory manner in the device.

### BACKGROUND OF THE INVENTION

Devices for the insertion of deformable intra-ocular lenses are known. In general, their principal purpose consists in being able to keep the incision in the eye necessary for the insertion of an intra-ocular lens as small as possible. A difficulty of such devices consists in bringing the intra-ocular lens into an elastically deformed state in such a manner that it can be injected in this state through a cannula into an eye. U.S. Pat. No. 4,681,102 shows a device of this type, wherein a lens holder comprises a hinge, which permits bringing the lens holder from an open state, in which the lens is laid in, into a closed state in which the lens is folded together. The hinge located in the center of the lens holder initially prevents the deformation of the lens and it can even happen that the lens relaxes at the beginning of the deformation process and is bent in the direction opposite to that intended. The device according to U.S. Pat. No. 5,947,975 provides an improvement in this regard by the lens holder comprising two hinges. Both of the devices mentioned above have the disadvantage that it is complicated to place the intra-ocular lens in the lens holder. An additional disadvantage of these devices consists in the fact that with them the intra-ocular lens is folded practically around a line oriented in the longitudinal direction of the device, which can lead to a spatial excess stress of the lens, whereby, under certain circumstances, it remains deformed long-term, in particular when it previously remained in the device too long.

### SUMMARY OF THE INVENTION

Proceeding from the state of the art the objective of the invention is to propose a device of the type stated in the introduction in which the intra-ocular lens can be brought into the deformed state in a simple and reliable manner and can be introduced into the device in this state.

To realize this objective the device is characterized by the fact that it comprises a lens holder which can be inserted into the device, that the lens holder comprises an elastic base which can be deformed in such a manner that it can be deformed starting from a relaxed, open position by bending into a stressed, closed position, where during the deformation the intra-ocular lens in contact with the lens holder is subjected to an increasing curvature.

According to one type of embodiment the device, the plunger comprises on its free end an indentation running essentially in the direction transverse to the cross section of the plunger. This is intended for the purpose of receiving an edge of the intra-ocular lens in order to be able to advance it reliably into and through the cannula.

According to another type of embodiment, the device comprises a bearing part for the lens holder, said bearing part being open towards the outside. The bearing part has the advantage that it can receive the lens holder, but also additional parts serving for precise positioning and guiding of the intra-ocular lens and the plunger, so that they fit exactly. Numbering among these additional parts can, for example, be an alignment device for the plunger, said alignment device in turn possibly comprising a guide element lying on the plunger. This alignment device can be connected to the bearing part as one piece or it can be removable. Even the cannula can be advantageously connected to the bearing part as one piece.

If, according to an additional type of embodiment, the lens holder does not project out of the bearing part, it is thereby prevented that the lens holder can be removed from the device after it has been inserted. This is desirable in the case of devices, which are intended for one-time use.

In an advantageous manner, the elastic base in the stressed position forms a channel in which the curved intra-ocular lens is located. According to one type of embodiment, this channel becomes narrower toward one side of the device, whereby the intra-ocular lens is compressed during its advance to the cannula.

If, according to an additional type of embodiment, the channel has a helical cross section at its end facing the cannula, larger intra-ocular lenses can be deformed in such a manner that their opposite edges overlap. The intra-ocular lens is thus rolled up in the device. According to another type of embodiment, this helical cross section can also be present in the passageway opening of the bearing part.

An additional type of embodiment provides that the elastic base has on its side facing the plunger a tapering in order to form a guide for the plunger. This tapering forms a ramp-like guide face for the plunger. Alternatively to this, a guide face of this type can be present on the alignment device.

According to still another type of embodiment, there are connecting means at the lens holder in order to hold the lens holder in its closed position. In addition, catching means can be present in order to position and to hold the lens holder in the device.

### BRIEF DESCRIPTION OF THE DRAWINGS

Embodiment examples of the invention, by way of example, are described in the following with reference to the accompanying drawings. Shown are:

FIG. 1 is a perspective view of an embodiment example of the device for the insertion of deformable intra-ocular lenses,

FIG. 2 is a perspective view of an embodiment example of the lens holder seen from the side of the plunger, and

FIG. 3 is a perspective view of another embodiment example of the lens holder seen from the side of the cannula.

### DETAILED DESCRIPTION OF PREFERRED EMBODIMENT

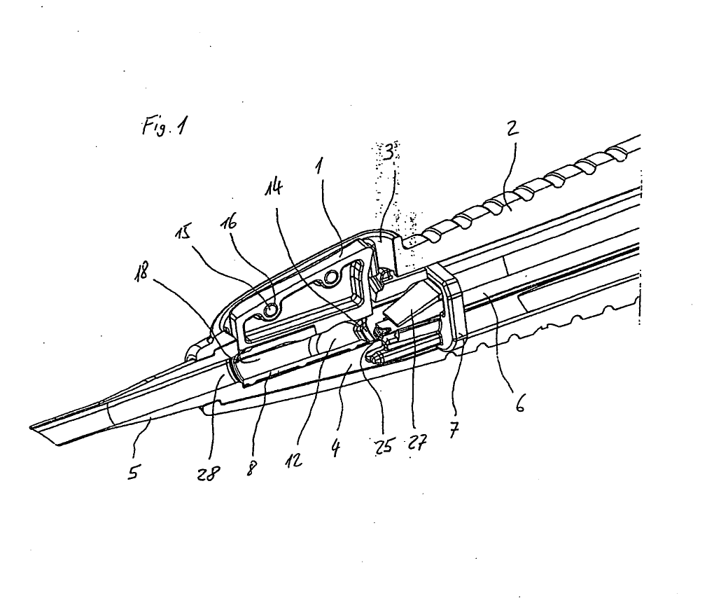

FIG. 1 shows an extract of a device for the insertion of deformable intra-ocular lenses with a lens holder 1 inserted in the device. The device comprises an elongated housing 2 in which an elongated opening 3 is provided which serves for the insertion of the lens holder 1. During the use of the device the lens holder 1 with an intra-ocular lens confined therein in the deformed state is inserted through the opening 3 into the device. Then the intra-ocular lens is advanced by a plunger 6 out of the lens holder 1 into a cannula 5. Subsequently, the cannula 5 is inserted though a small incision into the eye of a patient and the intra-ocular lens is pushed by the plunger 6 out of the cannula 5 into the eye.

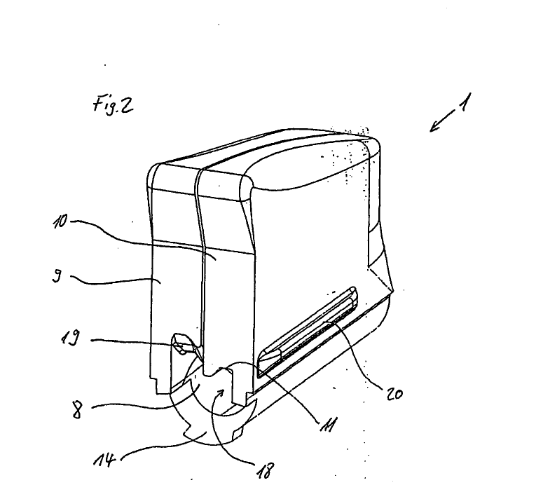

The lens holder 1 comprises an elastic base 8, which is essentially plane in the relaxed, open position. A tray 12 disposed in the elastic base 8 can simplify the placement of the intra-ocular lens. If an intra-ocular lens has been placed in the lens holder 1, said lens holder is deformed together with the intra-ocular lens lying on the elastic base 8 until it assumes the closed position represented in the figures. In this position the lens holder 1 forms a channel 18 running in the longitudinal direction of the device in which channel the deformed intra-ocular lens is located initially and but of which it is transported by the plunger 6 into a passageway opening 28 present in the cannula 5. So that the lens holder 1 remains in the closed position until insertion into the device, connecting means are provided which consist in the present example of studs 15 which are present at the edge area 10 of the lens holder 1 (FIG. 2) and engage in openings 16 which are disposed in the opposite edge area 9 (FIG. 2).

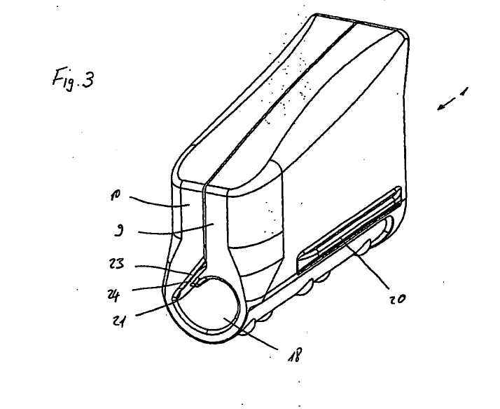

FIGS. 2 and 3 show additional details of types of embodiments of the lens holder 1. Said edge areas 9 and 10 are disposed on both sides of the elastic base 8. Along with this, the edge areas 9, 10 are thicker and thus more rigid than the elastic base 8. At the transition between the elastic base and each of the edge areas an undercut 11 is present which, during the deformation process, permits holding the edges of the intra-ocular lens to be laid in and guiding said edges during the displacement into the cannula 5. Known intra-ocular lenses comprise two so-called haptics. These are small retaining elements, which center the lens in the lens pocket in the eye. In order to insert such intra-ocular lenses with the lens holder according to the invention, the lens holder 1 advantageously has an expansion 19 (FIG. 2) in the area of the undercut 11, said expansion receiving a haptic and later during the displacement of the intra-ocular lens by the plunger 6 preventing said haptic from remaining hanging in the lens holder 1.

As FIG. 1 shows, the lens holder 1 is held in a bearing part 4 in the present example, where the latter, as represented, can be connected as one piece with the cannula 5. On at least one side of the closed lens holder, catching means are disposed, e. g. in the form of a rib 20 (FIGS. 2 and 3), which are intended to precisely position and to arrest the lens holder 1 in the housing 2 or in the bearing part 4. For reliable functioning of the device it is important that the plunger 6 engages the deformed intra-ocular lens exactly at one of its edges. For this, on the one hand the above-described means serve for precise positioning and arresting of the lens holder 1 in the device. On the other hand, various guide means for the plunger are provided which are described in the following.

An alignment device 7 provides for the plunger 6 assuming a definite position within the device. The plunger 6 is received in a passageway opening 29 of the alignment device 7 and is held by a guide element 27 in the position shown in FIG. 1. In the embodiment example represented, the alignment device 7 is connected by a type of plug-in connector to the bearing part 4. The alignment device 7 can however also be connected as one piece with the bearing part 4. The guide face 14 has the purpose of guiding the plunger 6 in such a manner that it strikes precisely the edge of the deformed intra-ocular lens if it should be pushed out of the lens holder 1 into the passageway opening 28 of the cannula 5. The guide face 14 can be formed, as represented, as a tapering on one side of the elastic base 8. However, as an alternative, the guide face can also be disposed on the bearing part 4. Also, on the plunger 6 itself means are provided which improve the precision of the engagement of the deformed intra-ocular lens. These means have the form of an indentation 25, which extends over the apical end of the plunger 6. Preferably, the indentation 25 runs in the form of an arc, where the radius of the arc corresponds to the bending radius of the deformed intra-ocular lens.

FIG. 3 shows a particular type of embodiment of the lens holder 1 which is provided especially for injecting relatively large intra-ocular lens. It is distinguished from that according to FIG. 2 by an embossing 21 which is disposed in the edge area 9 adjacent to the flexible base 8. In the opposite edge area 10 a corresponding recess is provided in which the embossing 21 is seated when the lens holder 1 is in the closed position. In FIG. 3 is can be seen clearly that the channel 18 is given a helical cross section on the exit side by the embossing 21. Along with this, between the embossing 21 and a guide part 23 there is also a gap 24 which permits a large intra-ocular lens to deform in such a manner that its opposite edges overlap, whereby the intra-ocular lens leaves the lens holder 1 in a rolled-up state. Alternatively, the helical form can also be formed in the passageway opening 28, which extends from the bearing part 4 through the cannula 5.

1-17. (canceled)

18. A device for inserting an elastically deformable intra-ocular lens into an eye, comprising:
a lens holder including an elastic base which is deformable from a relaxed, open position into a stressed, closed position, wherein the deformation of the elastic base increases a curvature of the intra-ocular lens disposed in the lens holder;
a cannula; and
a moveable plunger, wherein movement of the plunger pushes the elastically deformed intra-ocular lens from the lens holder through the cannula into the eye.

19. The device according to claim 18, wherein the plunger has a free end, and wherein the free end of the plunger has an indentation running essentially in a direction transverse to the cross section of the plunger, said indentation being configured to receive an edge of the intra-ocular lens.

20. The device according to claim 18, further including a bearing part for the lens holder, said bearing part being open towards the exterior of the device.

21. The device according to claim 18, further including an alignment device for the plunger.

22. The device according to claim 21, wherein the alignment device comprises a guide element on the plunger.

23. The device according to claim 21, wherein the bearing part and the alignment device are detachably connected.

24. The device according to claim 21, wherein the bearing part and the alignment device form one piece.

25. The device according to claim 20, wherein the bearing part and the cannula form one piece.

26. The device according to claim 20, wherein the lens holder does not project out of the bearing part.

27. The device according to claim 18, wherein the elastic base in the stressed position forms a channel in which the curved intra-ocular lens is located.

28. The device according to claim 27, wherein the channel formed in the stressed position becomes narrower toward one end of the channel.

29. The device according to claim 26, wherein that the channel has a helical cross section at its end facing the cannula.

30. The device according to claim 20, wherein the bearing part comprises a passageway opening for the intra-ocular lens, said passageway opening having a helical cross section on its side facing the channel of the lens holder.

31. The device according to claim 18, wherein the elastic base has on its side facing the plunger a tapering in order to form a guide for the plunger.

32. The device according to claim 18, wherein the alignment device comprises at its end facing the lens holder a guide face for the plunger.

33. The device according to claim 18, further comprising a connecting mechanism at the lens holder in order to hold the lens holder in its closed position.

34. The device according to claim 18, further comprising a catching mechanism for positioning and holding the lens holder in its position.

35. The device according to claim 19, further including an alignment device for the plunger.

36. The device according to 19, wherein the elastic base in the stressed position forms a channel in which the curved intra-ocular lens is located.

37. The device according to claim 36, wherein the channel formed in the stressed position becomes narrower toward one end of the channel.
